(19) United States
(12) Patent Application Publication
(10) Pub. No.: US 2019/0046985 A1
(43) Pub. Date: Feb. 14, 2019

(54) DROPLET-TRAPPING DEVICES FOR BIOASSAYS AND DIAGNOSTICS

(71) Applicant: The Regents of the University of California, Oakland, CA (US)

(72) Inventors: Dong-ku KANG, Irvine, CA (US); Weian ZHAO, Chicago, IL (US); Louai LABANIEH, Irvine, CA (US)

(21) Appl. No.: 15/759,752

(22) PCT Filed: Sep. 15, 2016
(86) PCT No.: PCT/US16/51964

§ 371 Date: Mar. 13, 2018

Related U.S. Application Data
(60) Provisional application No. 62/220,144, filed on Sep. 17, 2015.

(51) Int. Cl. B01L 3/00; G01N 33/58

(52) U.S. Cl. CPC B01L 3/502761; B01L 3/502784; G01N 33/582; B01L 2300/0816; B01L 2400/0478; B01L 2200/0673; B01L 2300/023; B01L 2200/0668

(57) ABSTRACT

In alternative embodiments, provided are high-throughput, multiplexed systems or methods for detecting a chemical, biological, a physiological or a pathological analyte, or a single molecule or a single cell in droplets using the floating droplet array system, whereby droplets are trapped in an array of trapping structures. In alternative embodiments, high-throughput, multiplexed systems as provided herein are integrated with portable imaging systems such as CCD, CMOS, digital camera, or cell phone-based imaging.

### STATEMENT AS TO FEDERALLY SPONSORED RESEARCH

This invention was made with government support under grant number RO1 AI117061-01, awarded by the National Institutes of Health (NIH), DHHS. The government has certain rights in the invention.

### TECHNICAL FIELD

Provided are compositions for encapsulated sample trapping, manipulation, analysis, sorting, and screening and methods for making and using the same.

### BACKGROUND

High-throughput technologies have found many applications in biology and chemistry such as drug discovery, disease diagnosis, and elucidating biological mechanisms. These applications often require detection of rare analytes such as nucleic acids, proteins, metabolites, and cells. In addition, these analytes often exist among a large background of interfering, non-target species. Moreover, real-time analysis is also often required to capture the dynamic nature of biological processes. Therefore, there is a great need for technologies that can isolate, analyze, and quantify individual components of a heterogeneous mixture in a parallel, high-throughput format. Traditional high-throughput technologies such as microwell plates with automated robotic handling systems are widely used in industries such as drug screening. However, these platforms require bulky, expensive machinery, are prone to sample evaporation, and require relatively large sample volumes, which can waste precious reagents or biological samples.

Recently, microfabricated devices have become powerful technologies for high-throughput analysis in many applications such as biological and chemical assays. These technologies often partition a bulk solution into many isolated pico to nanoliter-sized compartments. This compartmentalization confines rare analytes into a small volume, which increases their effective concentration and reduces interference from non-target species. This compartmentalization has been achieved using fluids dispersed into microfabricated wells or in microfluidic chambers which are separated by pneumatically controlled valves. However, retrieving individual samples from these types of devices is difficult to achieve. Moreover, reagent mixing requires complex architecture and microfabrication or is done in bulk before compartmentalization, which may prevent colocalization of initial reaction products from with their initiating target.

Another way to compartmentalize reactions is to partition them into discrete micron-sized droplets surrounded by an immiscible carrier fluid. Droplet-based microfluidics has the advantage of precise control over mixing of fluids, minimal waste of precious reagents, and reduces evaporation and adsorption of molecules at the device walls. Uniform droplets can be generated at kHz frequencies with sizes precisely controlled by fluid flow rates and device geometry. Multiple operations can be performed such as droplet fusion, splitting, cooling, heating, and sorting on- or off-chip as the application requires. Droplet microfluidic devices have been developed for a wide range of applications including micro-material fabrication, directed evolution, mRNA profiling of a heterogeneous population of cells, pathogen detection, and single-cell and single-molecule analysis.

Recently, Hatch and coworkers reported a high-throughput droplet digital PCR (ddPCR) device was developed that analyzed tightly packed droplets in a microfluidic chamber via an integrated CMOS-based wide-field imaging system for absolute quantification of copy number of target DNA. In this development, the dynamic range of ddPCR was increased by 100-fold compared to existing ddPCR systems by increasing the device throughput. However, droplet coalescence was observed for a small fraction of droplets, which was likely exacerbated by their tight-packing. Moreover, neighboring droplets which are close together or overlap complicate image processing, which may result in quantification errors in this type of device. Indexing poses an additional challenge since droplets are free to move throughout the experiment, which hinders real-time monitoring.

Spatially defined arrays of static, immobilized droplets facilitates indexing and monitoring of droplets over time since the array element locations create a natural positioning system. Recently, Huebner and colleagues used droplet traps to immobilize droplets into a 384 element array, which allowed for monitoring of the droplets over time. The droplets could also be subsequently recovered by reversing the flow direction. Similarly, Schmitz and coworkers used channels containing many constrictions to trap up to 8000 droplets. Droplets were subsequently recovered by increasing the flow rate through the channels. However, ultrahigh-throughput analysis is difficult to achieve in these types of devices because the trapping structures are located within the main flow stream and thus a high-density of droplet traps results in a large resistance to flow. Moreover, they are prone to reagent and sample waste since the majority of the droplets pass around the traps. Microfluidic devices have also been previously reported that trap droplets by buoyancy forces between the drops and the carrier fluid. However, these devices require precise alignment of PDMS layers and the highest throughput achieved was only 120 droplet traps, which is comparably low-throughput and impractical for many biological applications. Thus, new devices are needed that can more precisely control droplet trapping, manipulation, analysis, and recovery in an efficient, ultrahigh-throughput manner.

### SUMMARY

In some embodiments, microfluidic droplets are trapped into an array of trapping structures. In some embodiments, droplets are trapped by buoyancy forces between immiscible fluids having different densities. In some embodiments, the droplets can be recovered from the trapping structures by reorienting the device. In some embodiments, various shapes and sizes of trapping structures may be used depending on the application such as droplet trapping, droplet incubation, droplet merging, droplet splitting, sample transfer, and buffer exchange between droplets. In alternative embodiments, these operations are conducted in a massively parallel and high-throughput manner.

In alternative embodiments, provided are guiding structures such as tracks, pillars, or narrow channels which guide droplets to the trapping structures and ensure complete coverage of the trapping structures. In alternative embodiments, inlets or outlets are included to divert droplets away or to the droplet trapping structures and/or other channels or chambers. In some embodiments, products of manufacture as provided herein are integrated with various imaging systems such as a fluorescence microscope, embedded avalanche photodiode (APD), photomultiplier tube (PMT), digital camera, charge-coupled device (CCD), or complementary metal-oxide semiconductor (CMOS) sensor for endpoint or real-time analysis. In some embodiments, products of manufacture as provided herein are integrated with droplet generation modules, droplet trapping modules, droplet manipulation modules, droplet recovery modules, and droplet analysis (e.g., imaging) modules. In some embodiments, products of manufacture as provided herein are packaged in fully integrated, automated, portable systems (see, for example, a non-limiting embodiment depicted in FIG. 12). In some embodiments, the device is integrated with data acquisition hardware and software, data processing software, display screens, and a user interface. In some embodiments, products of manufacture as provided herein are synched or integrated with digital communication and computer or mobile device applications. In some embodiments, products of manufacture as provided herein are used in rapid and sensitive assays for detecting and quantifying a chemical, biological, physiological, or pathological analyte, or a single molecule or a single cell.

In alternative embodiments, provided are high throughput, multiplexed systems or devices, or methods, for detecting and/or quantifying a chemical, biological, physiological or pathological analyte, or a single molecule or a single cell using a floating droplet array (FDA) system integrated with use of a sensing element, comprising:

(a) providing a sensor or sensing reaction that involves a small molecule, peptide, protein, nucleic acid, enzyme, antibody, cell, or chemical agent capable of detecting a target of interest.

(b) providing a floating droplet array system, droplet microfluidics system or microdroplet-manipulating device or system

(d) providing a chemical, biological or an environmental sample containing a target of interest such as a small molecule, metabolite, peptide, protein, nucleic acid, or cell

(e) encapsulating the chemical, biological or environmental sample into a plurality of microdroplets, trapping the microdroplets into trapping structures, and processing the microdroplets comprising the encapsulated chemical, biological or environmental sample in the droplet microfluidics system or microdroplet-manipulating device, and detecting the presence of a fluorophore signal, or fluorescence, in each microdroplet in the device

wherein detection of a fluorophore signal or fluorescence in a microdroplet indicates the presence of the target molecule in the microdroplet, and the sample.

In alternative embodiments, provided are high throughput, multiplexed systems or devices, or methods, for detecting and/or quantifying a chemical, biological, a physiological or a pathological analyte, or a single molecule or a single cell using a floating droplet array system in real-time.

In alternative embodiments, provided are high throughput, multiplexed systems or devices, or methods, for detecting and sorting of single molecules of chemical, biological, a physiological or a pathological analytes, or a single molecule or a single cell using a floating droplet array system.

In alternative embodiments, the cell is a mammalian cell, a circulating tumor cell, a circulating melanoma cell, fungal cell, virus or a bacterial cell.

In alternative embodiments, the droplet microfluidics system can generate: picoliter droplets or droplets of between about 2 μm to 999 μm in diameter (including any diameter size in between and including these endpoints). In alternative embodiments, 100 to 100 billion droplets can be immobilized and analyzed in the droplet array.

In alternative embodiments, droplets are composed of one or many sub-phases as single or multiple emulsions.

In alternative embodiments, a biological sample comprises a blood, serum, saliva, tear, urine, tissue, or CSF sample (or other biological fluid, or sample derived from a non-fluid starting sample, such as a tissue homogenate) from a patient as well as non-biological samples including food, water and environmental samples.

In alternative embodiments, the single molecule is a nucleic acid, a nucleic acid point mutation, or a single-nucleotide polymorphism (SNP), ribonucleic acid or a nucleic acid biomarker for, e.g., breast cancer. In alternative embodiments, the single molecule is a protein, a lipid, a carbohydrate, a polysaccharide, a small molecule or a metal. In alternative embodiments, the single cell is a bacteria fungi, virus, and mammalian cells.

In alternative embodiments, the aptamer is an oligonucleotide, a nucleic acid or a peptide aptamer. In alternative embodiments, the sensor comprises a DNA strand displacement strategy, a proximity ligation assay, or a binding induced DNA assembly assay, or equivalents.

In alternative embodiments, high throughput, multiplexed systems or devices, or methods, as provided herein further comprise disposable microfluidic “cartridges,” permitting multiplex and rapid detection of multiple types of targets simultaneously, and optionally the high throughput, multiplexed system or device is fully automated, or is fabricated as an all-in-one system or with modular components, or is linked (e.g., by wired or wireless linkage, such as Bluetooth) to an electronic device, e.g., a portable device, e.g., a smart phone and/or a tablet, laptop, for point-of-care applications.

In alternative embodiments, the throughput, multiplexed system is engineered to comprise one or any of: desirable portability (for example, packaged as backpacks), automating fluid handing (i.e., droplet generation and auto sampling), and integrating electronics including a diode laser, LED panel, light source, operating, and/or data analyzing software, display with fluorescence microscopy, embedded APD (avalanche photodiode), photomultiplier tube (PMT), digital camera, charge-coupled device (CCD), complementary metal-oxide semiconductor (CMOS) sensor.

In alternative embodiments, high throughput, multiplexed systems or devices, or methods, as provided herein further comprise disposable microfluidic “cartridges,” permitting multiplex and rapid detection of multiple types of targets simultaneously, and optionally the high throughput, multiplexed system or device is fully automated, or is fabricated as an all-in-one system or with modular components, or is linked to an electronic device, e.g., a portable device, e.g., a smart phone and/or a Bluetooth, for point-of-care applications.

In alternative embodiments, high throughput, multiplexed systems or devices, or methods, as provided herein further comprise, or comprise, trapping structures of various sizes/shapes for immobilizing droplets of various compositions in a spatially controlled, defined, and parallel format. In alternative embodiments, the high-throughput system traps droplets into trapping structures, whereby droplets float or sink into trapping structures due to density differences between the dispersed and continuous phases. In alternative embodiments, the droplets may be recovered by reorienting the device.

In alternative embodiments, a high-throughput system as provided herein comprises a multilayer microfluidic device whereby droplets are trapped in a region above or below the main flow stream. In alternative embodiments, the high-throughput system can comprise guiding structures such as tracks, pillars, or narrow channels which guide droplets to the trapping structures and ensure complete and efficient coverage of the trapping structures. In alternative embodiments, the high-throughput system comprises inlets or outlets to divert droplets away or to the droplet trapping structures and/or other channels or chambers.

In alternative embodiments, a high-throughput system as provided herein is integrated with sorting elements for retrieving many or individual droplets, whereby the sorting elements may be electrode, pneumatic valve, laser, microneedle, or acoustic-based droplet retrieval systems. In alternative embodiments, a high-throughput system as provided herein can index droplets based on one or many spatial or temporal variables. In alternative embodiments, droplets are indexed based on uniquely barcoded beads, nucleic acid barcode, fluorophore, or colorimetric barcode.

In alternative embodiments, a high-throughput system as provided herein is integrated with data acquisition hardware or software, data analysis software, a user interface, or computer or mobile device applications.

In alternative embodiments, provided are high-throughput droplet generation modules, whereby droplets are formed at high throughput using droplet-generating junctions comprising:
(a) a dispersed phase which flows through a plurality of channels in a radial direction prior to dispersion.
(b) a carrier phase that flows through one or many channels in a direction perpendicular to the radially flowing phase that is to be dispersed.
(c) droplets generated as the carrier phase comes into contact with the immiscible dispersed phase in the arrangement described in (a) and (b).

In alternative embodiments, a high-throughput system as provided herein comprises a stacked, 3D arrangement of droplet-generating junctions such that droplet generation can occur at many junctions simultaneously.

In alternative embodiments, a high-throughput system as provided herein comprises droplet-generating module integrated within a portable handheld fluidic device, such as a syringe.

In alternative embodiments, a high-throughput system as provided herein comprises a driving pressure for fluid flow, which can be generated by hand using a force-transferring device such as a plunger.

The details of one or more embodiments as set forth in the accompanying drawings and the description below. Other features, objects, and advantages of alternative embodiments will be apparent from the description and drawings, and from the claims.

All publications, patents, patent applications cited herein are hereby expressly incorporated by reference for all purposes.

### DESCRIPTION OF DRAWINGS

Figures are described in detail, below. Like reference symbols in the various drawings indicate like elements, unless otherwise stated.

FIG. 1. A schematic illustration for the workflow of the Floating Droplet Array. (a) General workflow involves droplet generation, trapping for analysis, and subsequent droplet recovery (b) Step-by-step operation: (i) generated droplets flow into the trapping chamber and float into the wells; (ii) after all the wells have been filled, (iii) the remaining droplets are purged; and (iv) the trapped droplets are then analyzed (v) droplets are recovered by flipping the device so that droplets float out of the wells and (vi) droplets are sent for downstream handling on- or off-chip.

FIG. 2. Images depicting the workflow for the Floating Droplet Array. (a) Photographic image of entire microfluidic device. The device was filled with green dye for visualization, scale bar=1 cm; (b) Schematic representation of the workflow including (i) droplet generation, (ii) droplet loading into the chamber, (iii) droplet trapping, (iv) filling the chamber, (v) purging extraneous droplets, and (vi) droplet recovery by flipping. Blue arrows in (ii)-(vi) represent flow direction. All scale bars for (b)=200 μm.

FIG. 3. CAD rendering of the Floating Droplet Array. The top layer of the FDA device contains the droplet-trapping microwells while the bottom layer contains the droplet generation and chamber modules (middle panel). Droplets are generated using a flow-focusing structure (Left panel) and trapped into circular microwells (Right panel). Device geometries were exaggerated in the rendering for visualization purposes.

FIG. 4. Device design parameters for efficient droplet trapping and recovery.

FIG. 5. Microscopic images of the ultrahigh-throughput FDA using 50 μm wells. (a) Large-scale scan of 36 images containing more than 14,000 wells and (b, c) zoomed-in images of trapped droplets. Scale bar=75 μm.

FIG. 6. Controlling the number of droplets per well. (a) one droplet per well, (b) two droplets per well, (c) three droplets per well and (d) four droplets per well. Scale bars (a-b)=120 μm. (e) Controlling droplet size by manipulating the flow rate ratio (water/oil) for 30×50 μm channel (circles) and 15×50 μm channel (squares).

FIG. 7. Droplet crosstalk studies of the diffusion of fluorescein between clustered droplets. (a) Hydrolysis of FDG by β-gal. (b) Microscopic image showing overlay of bright-field and FITC channels for FDG droplets with and without a β-gal bead after a 4 hour incubation, scale bar=50 μm. (c) Fluorescent microscopic images showing time course of reaction over a 4 hour incubation to monitor droplet crosstalk, scale bar=50 μm. (d) Fluorescent intensity of trapped droplets with bead (back row), without bead (middle row), and the oil phase (front row).

FIG. 8. Digital quantification of the number of droplets containing a β-gal bead. (a) Microscopic image of trapped droplets in a device containing 109,569 microwells of 30 μm size. Droplets are generated with 250 μM FDG and a low concentration of β-gal beads so that most droplets do not contain any beads. Insert depicts a zoomed bright field microscopic image of a bead-containing droplet. White circle highlights a β-gal bead (7.8 μm) within a droplet. (b) Fluorescence microscopic images of droplets within 30 μm microwells. 0=without target bead (dark droplet without β-gal bead), 1=with bead (fluorescent droplet with β-gal bead). All scale bars=200 μm.

FIG. 9. Encapsulation and single bacteria detection using the floating droplet array device. Single β-lactamase-producing bacterial cells were encapsulated within picoliter-sized droplets (55 μm) along with a fluorogenic substrate for beta-lactamase. The fluorescent intensity was monitored after a 4 hour incubation. Scale bar=120 μm.

FIG. 10. Quantification of fluorescent droplets. The digital single lens reflex (dSLR) camera can be used to quantify fluorescence droplet. LED or LCD panels can be used as an excitation light source.

FIG. 11. Digital quantification of fluorescent droplets from the floating droplet array device using a CMOS sensor. CMOS sensor can be used to monitor emitted light to analyze droplets for digital quantification. LED or LCD panels can be used as an excitation light source.

FIG. 12. An illustration of a portable floating droplet array system.

FIG. 13. Various exemplary shapes for droplet trapping structures in the floating droplet array (side view). Example shapes include rectangles, semicircles, triangles, or trapezoids.

FIG. 14. Various exemplary shapes for droplet trapping structures (top view). Example shapes include rectangles, circles, pentagons, stars, triangles, or cross shapes.

FIG. 15. Spatial patterning of trapping structures. The size of and distance between microwells can be varied. For example, parameters X, Y, and Z can be varied.

FIG. 16. Exemplary parameters for droplet trapping structures and chamber. The size (depth and width) of the microwells and height of chamber can be varied. For example, X, Y, and Z can be varied.

FIG. 17. Sized-based clustering of droplets (workflow). Different sizes and shapes of microwells can be fabricated to cluster multiple droplets according to their size. Large droplets can be generated first and trapped in respective large trapping structures. Smaller droplets can then be generated and trapped into respective smaller trapping structures.

FIG. 18. Sized-based clustering of droplets (side and top views). Different sizes and shapes of microwells can be fabricated to cluster multiple droplets according to their size. Large droplets can be generated first and trapped in respective large trapping structures. Smaller droplets can then be generated and trapped into respective smaller trapping structures.

FIG. 19. Manipulation of mass diffusion between droplets and droplet fusion using chemical means. Clustered droplets can be induced to fuse or increase the diffusion of molecules between droplets using chemical reagents such as an alcohol solution (e.g., 2,2,3,3,4,4,4-Heptafluoro-1-butanol).

FIG. 20. Manipulation of mass diffusion between droplets and droplet fusion using physical means. Clustered droplets can be induced to fuse or increase the diffusion of molecules between droplets using integrated metal or solution-based electrodes.

FIG. 21. Selective droplet recovery using optics. Trapped droplets can be precisely manipulated using lasers to release selected droplet from trapping structures). Example laser-based manipulation include optical tweezers or microtsunami (laser-based microcavitation bubbles).

FIG. 22. Selective droplet recovery using pneumatic valves. Trapped droplets can be precisely manipulated using pneumatic valves to release droplets from trapping structures.

FIG. 23. High-throughput droplet generation module.

FIG. 24. High-throughput droplet generation module using 3D structured droplet generators.

FIG. 25. Syringe-based, high-throughput droplet generator.

### DETAILED DESCRIPTION

Provided are high-throughput platforms to manipulate, analyze, and screen microfluidic droplets in a parallel format. Droplet trapping is achieved at high efficiency and throughput by trapping droplets in a secondary layer away from the main flow stream. This format allows for the trapping of up to millions or billions of droplets in areas ranging from 1 mm² to 1 m² (or any area between, and including, these endpoints). Droplets trapped in a spatially-defined array facilitates droplet indexing since the array element locations provide a natural positioning system. This is particularly useful for monitoring a process over time or synchronizing reactions from a plurality of droplets to initiate simultaneously. In some embodiments, droplets are passively trapped into the trapping structures by buoyancy forces due to differences in densities between the discrete and carrier phases. In some embodiments, the trapped droplets can be recovered by reorienting the device and sent downstream for further processing on- or off-chip.

Provided are compositions and methods for trapping and analyzing droplets containing a sample at high-throughput and in a parallel format. This may be used in chemical and biological assays for the detection of metabolites, small molecules, proteins, lipids, nucleic acids, viruses and cells. In some embodiments, detection of analytes can be achieved by using sensor elements. In some embodiments the sensor elements are comprised of oligonucleotides, peptides, proteins, aptamers, antibodies, and cells. In some embodiments, signal amplification reactions such as polymerase chain reaction (PCR), reverse transcriptase PCR (RT-PCR), loop-mediated amplification reaction (LAMP), exponential amplification reaction (EXPAR), rolling circle amplification (RCA), strand displacement amplification (SDA), hybridization chain reaction (HCR), nucleic acid sequence based amplification (NASBA), helicase dependent amplification (HDA), nicking enzyme amplification reaction (NEAR), recombinase polymerase amplification (RPA), and enzymatic reaction may be used. In alternative embodiments, the devices are integrated with temperature-controlling systems (from 4° C. to 95° C.) and with heating and cooling functions so reactions in droplets can be controlled at desirable temperatures.

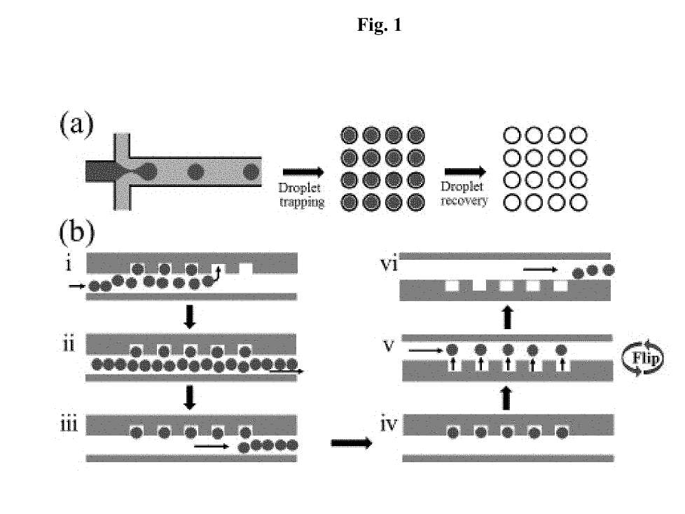

In alternative embodiments, exemplary platforms or systems as provided herein enable rapid and simple droplet manipulation using a floating droplet array system (e.g., as schematically illustrated in FIG. 1, a schematic illustration for the workflow of the Floating Droplet Array. (a) General workflow involves droplet generation, trapping for analysis, and subsequent droplet recovery (b) Step-by-step operation: (i) generated droplets flow into the trapping chamber and float into the wells; (ii) after all the wells have been filled, (iii) the remaining droplets are purged and (iv) the trapped droplets are then analyzed v) droplets are recovered by flipping the device so that droplets float out of the wells and (vi) droplets are sent for downstream handling on- or off-chip.

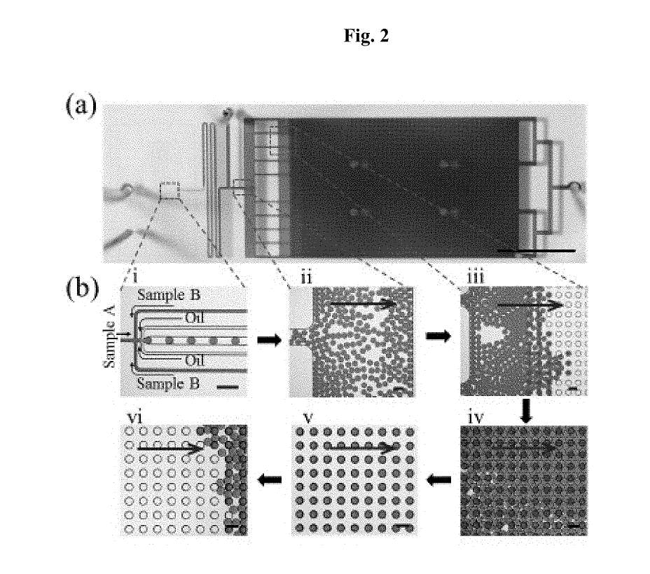

We demonstrated the effectiveness of an exemplary system as provided herein by generating, trapping, and recovering droplets. FIG. 2 shows the workflow for the Floating Droplet Array. (a) Photographic image of the entire microfluidic device. The device was filled with green dye for visualization, scale bar=1 cm; (b) Schematic representation of the workflow including (i) droplet generation, (ii) droplet loading into the chamber, (iii) droplet trapping, (iv) filling the chamber, (v) purging extraneous droplets, and (vi) droplet recovery by flipping. Blue arrows in (ii)-(vi) represent flow direction. All scale bars for (b)=200 μm

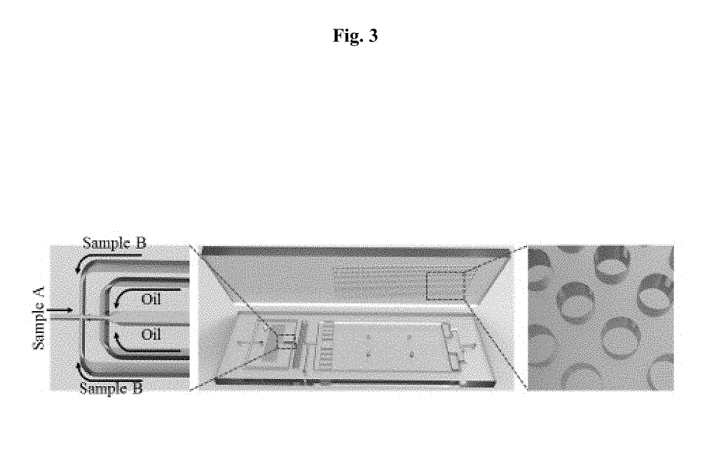

A CAD rendering of the Floating Droplet Array is shown in FIG. 3. The top layer of the FDA device contains the droplet-trapping microwells while the bottom layer contains the droplet generation and chamber modules (middle panel). Droplets are generated using a flow-focusing structure (Left panel) and trapped into circular microwells (Right panel). Device geometries were exaggerated in the rendering for visualization purposes.

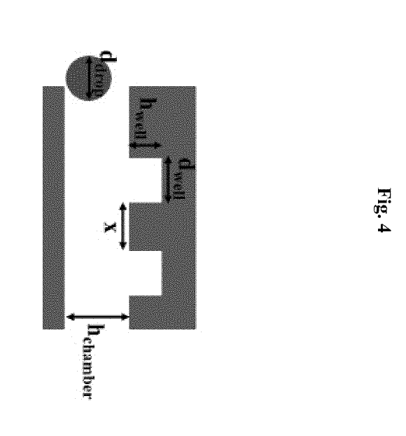

In alternative embodiments, geometric parameters as provided herein, such as the diameter of the well, d_{well}, depth of the well, h_{well}, height of the chamber, h_{chamber}, and inter-well spacing, x, can be chosen accordingly to efficiently trap, manipulate, analyze, and release a droplet in a well (FIG. 4). All the parameters can be varied according to the application.

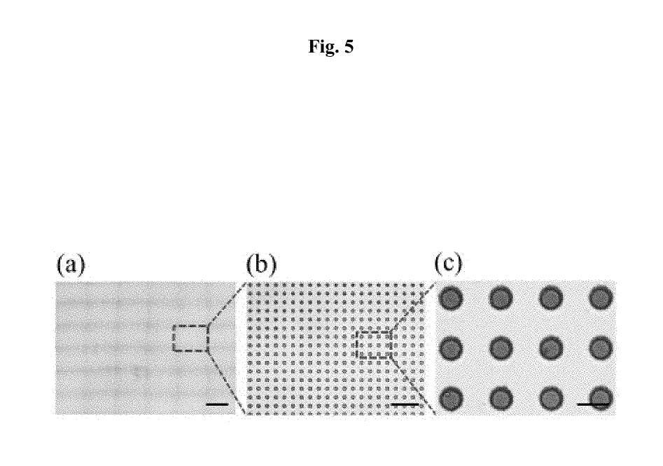

FIG. 5 shows an ultrahigh-throughput floating droplet array. (a) Large-scale scan of 36 images containing more than 14,000 wells and (b, c) zoomed-in images of trapped droplets (scale bar in (c) is 75 um). Moreover, the device is highly efficient in trapping droplets as can be seen in FIG. 5 with 100% of >14,000 wells analyzed containing a single droplet. We found that with the device dimensions used in this study (which are merely a representative example and non-limiting, as other dimensions can readily be used), we can consistently fill 100% of the microwells with single droplets when they are generated to be 10-20% smaller in diameter compared to the microwells.

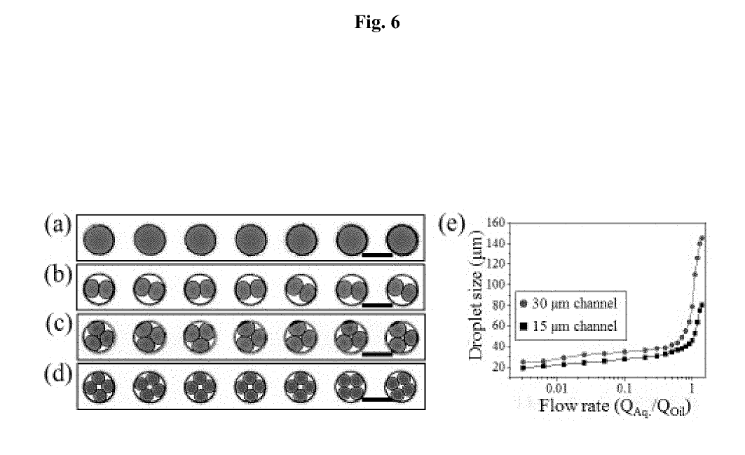

In alternative embodiments, exemplary platforms or systems as provided herein can be used for multiple droplet clustering into a single trapping structure in a simple, robust, and well-controlled manner. This can be achieved by varying the size of the droplets so that more than one droplet could fit into each well. As seen in FIG. 6, we demonstrated to precisely manipulate one, two, three, and four droplets per well by controlling the droplet size. This ability of the FDA device can be used for clustering multiple droplets that contain different samples or reagents within the same microwell for various complex biological studies such as enzymatic assays, drug screening, and cell-cell communication. This can be achieved by controlling diffusion (crosstalk) between droplets or merging droplets within the same microwell, in a highly parallel manner. FIG. 6 shows the manipulation of the number of droplets per well by varying droplet size. (a) one droplet per well, (b) two droplets per well, (c) three droplets per well and (d) four droplets per well. Scale bars (a-b)=120 (e) Controlling droplet size by manipulating the flow rate ratio (water/oil) for 30×50 μm channel (circles) and 15×50 μm channel (squares).

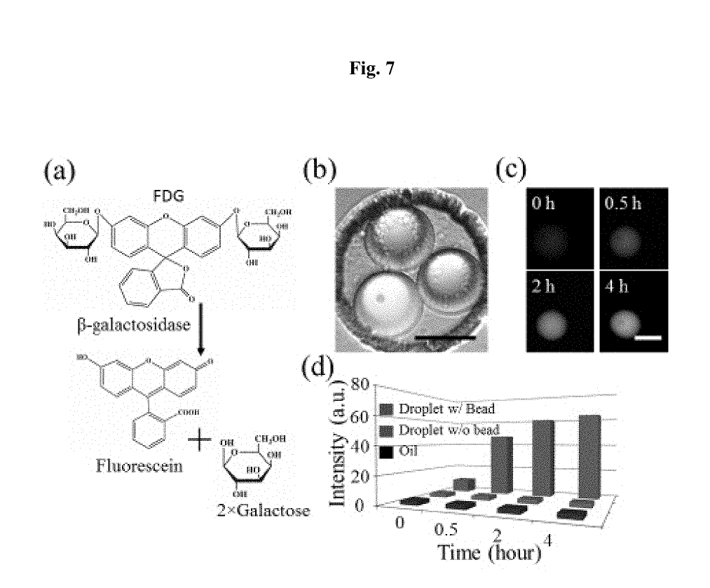

In alternative embodiments, systems as provided herein can be used to monitor diffusion of agents between droplets within the same microwell as shown in FIG. 7. To study this phenomenon using our FDA device, we chose β-galactosidase (β-gal) and its fluorogenic substrate (FDG) as a model system (FIG. 7a). We encapsulated 250 μM FDG and a low concentration of β-gal-conjugated microbeads, which resulted in only a few droplets containing a β-gal-conjugated bead and most droplets containing FDG without any β-gal beads.

In alternative embodiments, systems o as provided herein can be used to cluster multiple droplets of differing contents (i.e. cells, reagents, or samples) and merging or splitting them using a chemical reagent or externally applied electric field.

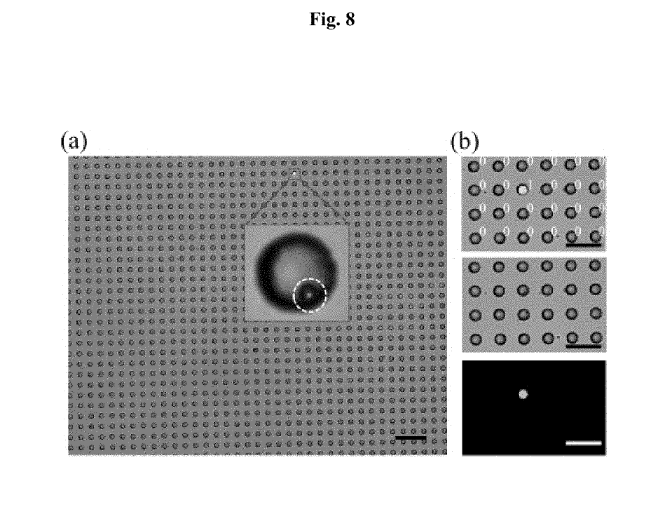

In alternative embodiments, exemplary platforms or systems as provided herein can be used for digital quantification of single molecules. We demonstrated digital quantification of analytes with spatially indexed droplets. This was achieved by encapsulating FDG along with a very low concentration of β-gal beads (10 beads/μl) so that the majority of droplets contain no β-gal bead and only a few droplets contain only one bead. Streptavidin-conjugated beads (7.8 μm) were used since they can be easily visualized and can also immobilize a large number of β-gal molecules, to yield strong enzymatic activity. As can be seen in FIG. 8a, there is only one fluorescent droplet, and it is the only one that contains a β-gal bead, among 1008 droplets in the image.

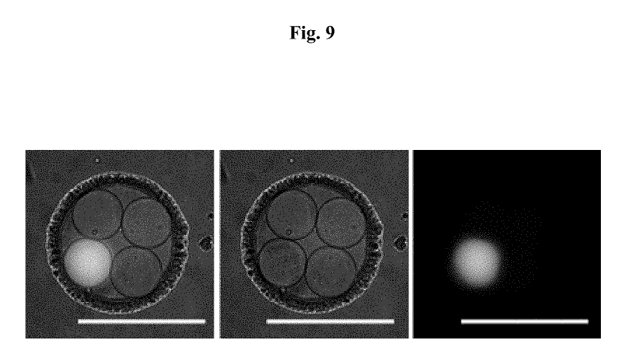

In alternative embodiments, products of manufacture as provided herein are used for monitoring single cells. In alternative embodiments, products of manufacture as provided herein are used for monitoring antimicrobial-resistant bacteria. We demonstrated encapsulation and detection of single bacteria using the floating droplet array device in FIG. 9. β-lactamase producing bacterial cells were encapsulated within picoliter-sized droplets (55 μm) at the single-cell level along with fluorogenic substrate for β-lactamase. The fluorescent intensity was monitored after 4 hour incubation (FIG. 9). Scale bar=120 μm.

In alternative embodiments, products of manufacture as provided herein can immobilizing droplets in a manner that yields facile indexing of droplets that is needed for real-time monitoring over an extended period of time. For example, it can be used for many applications such as single-cell or molecule analysis, genetic sequencing, biochemical profiling, cell culture, pathogen detection, and drug discovery.

In alternative embodiments, a floating droplet array system as provided herein can be integrated with various functions for further manipulating droplets such as droplet splitting and fusing in parallel or sequential formats.

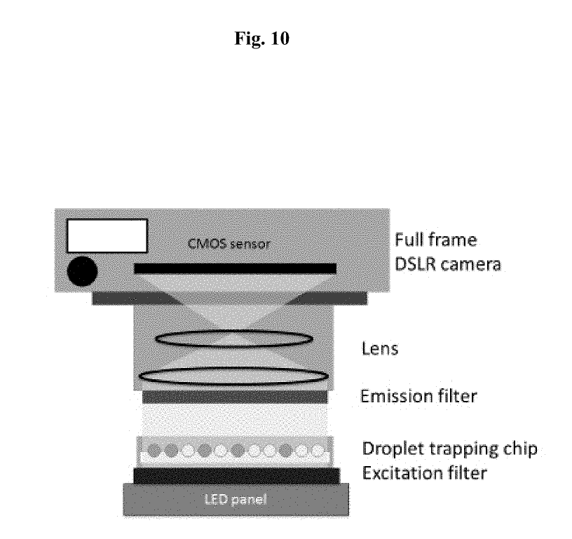

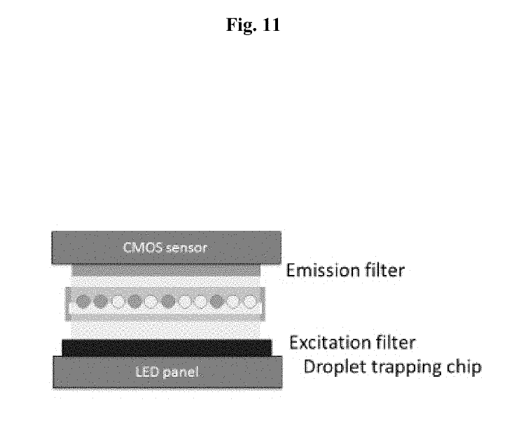

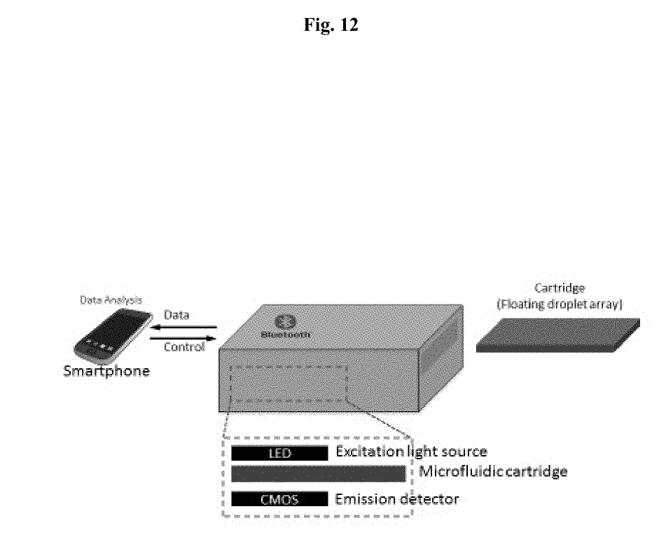

In alternative embodiments, a floating droplet array system as provided herein can be used for portable, point-of-care technologies when combined with CMOS, CCD, or cell phone-based imaging systems. FIG. 10 and FIG. 11 shows a schematic illustration of digital quantification of fluorescent droplets from the floating droplet array device. A digital single lens reflex (dSLR) camera (FIG. 10) or CMOS sensor can be used to quantify fluorescence droplets. LED or LCD panels can be used as an excitation light source. FIG. 12 is a non-limiting illustration of one embodiment of a portable floating droplet array system that can be used for point-of-care or portable diagnostics and integrated with a smartphone or tablet PC.

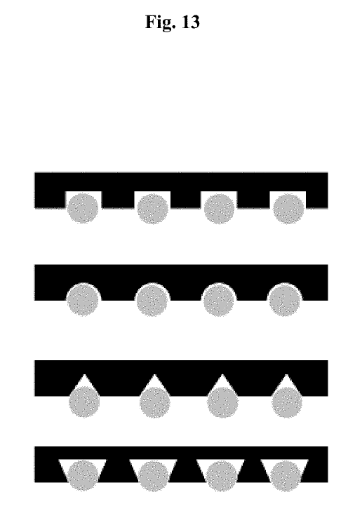

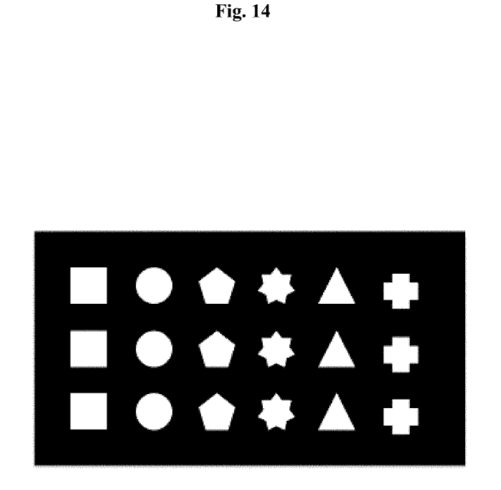

In alternative embodiments of a floating droplet array system as provided herein, the shape of the droplet trapping structures can be varied to form any shape such as a rectangle, semicircle, triangle, or trapezoid (FIG. 13 and FIG. 14).

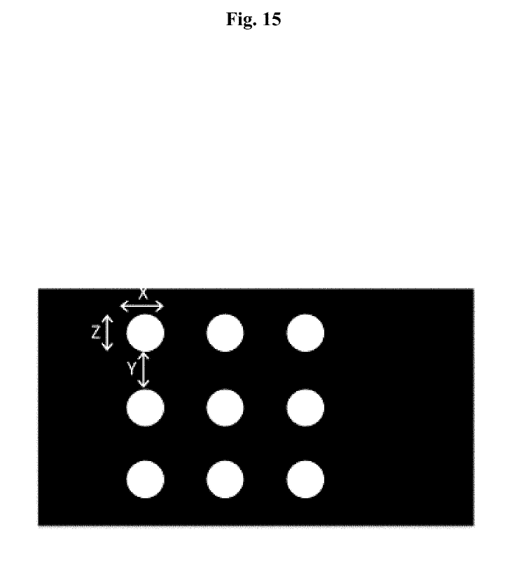

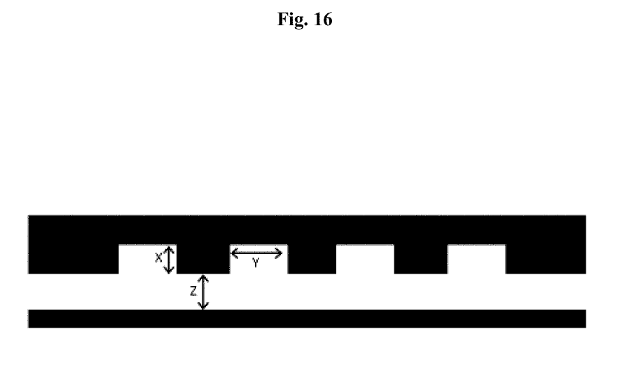

In alternative embodiments, of a floating droplet array system as provided herein, the size and spacing of the droplet trapping structure can be varied as can be seen in FIGS. 15 and 16. Example parameters of X, Y and Z can be varied.

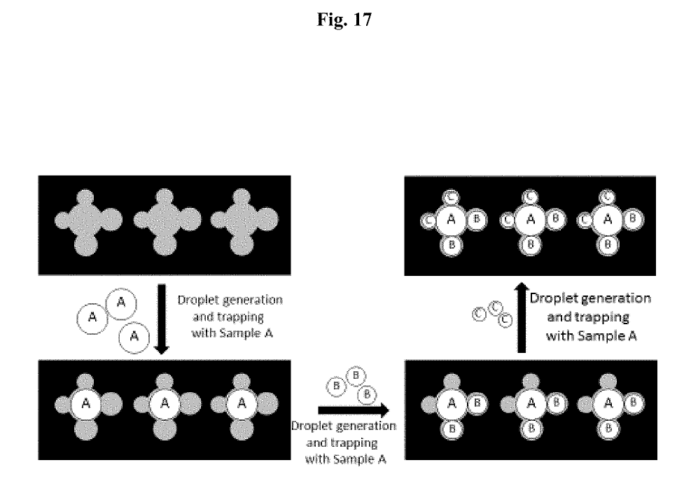

In alternative embodiments, of a floating droplet array system as provided herein can be used for sized-based clustering of multiple floating droplets in an array format (FIG. 17). Different-sized microwells can be fabricated to cluster droplets in a well-controlled, parallel manner according to their size. Bigger droplets are generated first and can be trapped in their respective, relatively large microwells. Smaller droplets are then generated and are immobilized into their respective microwells. This process can be continued in this manner to precisely control the arrangement and content of clustered droplets.

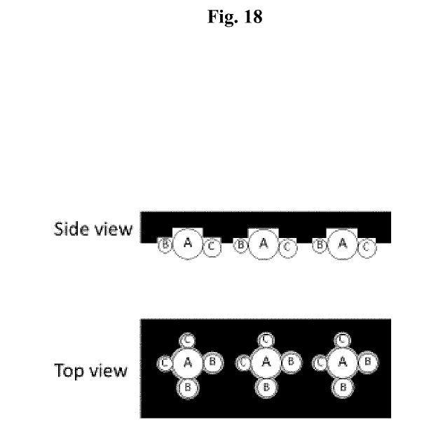

For example, sample A can be encapsulated into bigger droplets, sample B can be encapsulated in middle-sized droplets, and sample C can be encapsulated into small droplets as in FIG. 17 or FIG. 18.

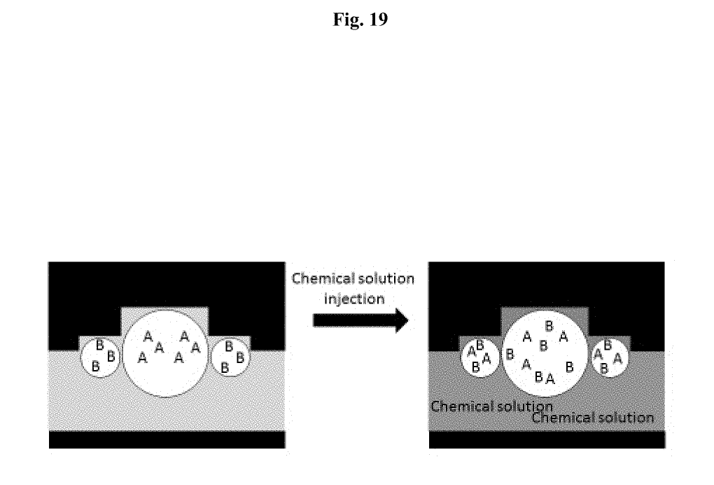

In alternative embodiments, droplet diffusion and droplet fusion as provided herein can be manipulated through physical (e.g., applied electric field) and chemical (e.g. reagents such as an alcohol solution (e.g., 2,2,3,3,4,4,4-Heptafluoro-1-butanol)) means. FIG. 19 shows manipulation of droplet diffusion and droplet fusion. Clustered droplets can be induced to fuse or increase mass diffusion between neighboring droplets using chemical reagents.

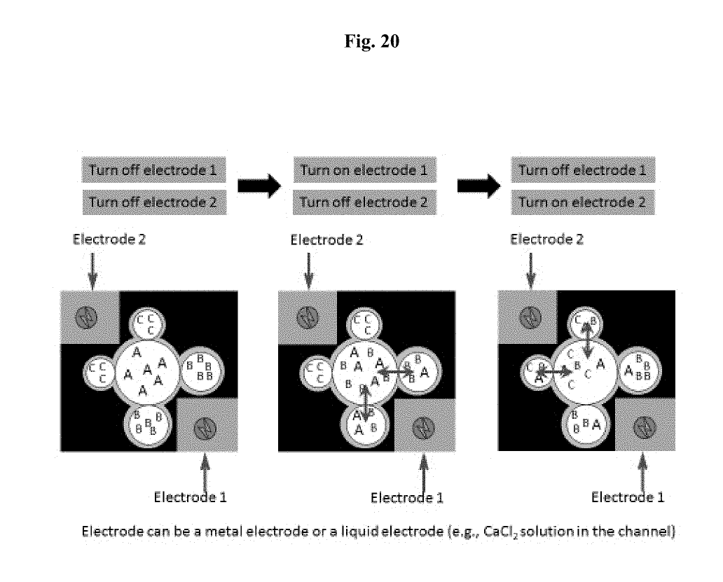

In alternative embodiments, a clustered floating droplet array as provided herein can be manipulated by an externally applied electric field. FIG. 20 shows manipulation of droplet diffusion and droplet fusion by externally applied electric fields. Clustered droplets can be induced to fuse or increase mass diffusion between neighboring droplets using an electric field applied via metal or solution-based electrodes.

In alternative embodiments, relatively large droplets are encapsulated with one or more cells and trapped into respective large microwells. Smaller droplets containing cell nutrient media, chemical reagents, biomolecules, beads, or cells can then be generated and clustered with the large droplets by trapping into respective microwells. Diffusion or fusion between droplets may or may not be manipulated using a chemical reagent, electric or magnetic field, or thermal or optical radiation.

In alternative embodiments, a product of manufacture as provided herein can be used for fabrication of complex heterogeneous composite materials. For example, monomer A can be encapsulated into bigger droplets, monomer B can be encapsulated in middle-sized droplets, and monomer C can be encapsulated into small droplets. The droplets can then be precisely assembled through size-based clustering. Subsequently, the droplets can be polymerized by the addition of a chemical reagent, light or thermal radiation to yield a composite material with isotropic or anisotropic properties.

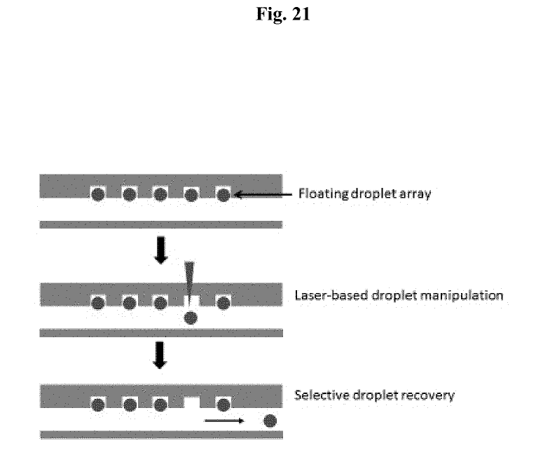

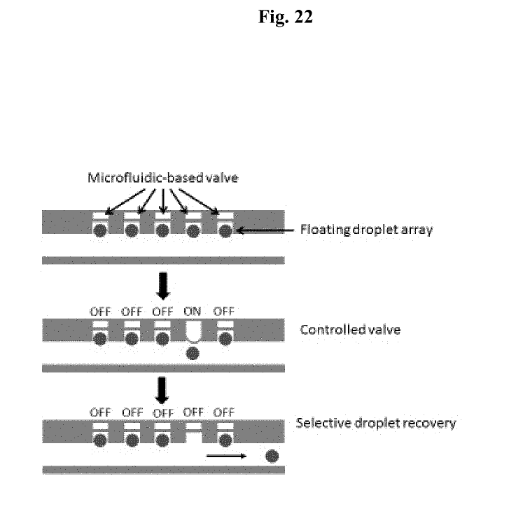

In alternative embodiments, a clustered floating droplet array as provided herein can be used to selectively sort/isolate and correspondingly recover droplets. FIG. 21 shows manipulation of selective droplet recovery. Trapped droplets can be precisely manipulated using optics to release selected droplet from trapping structures. Example laser-based manipulation include optical tweezers or microtsunami (laser-based microcavitation bubbles). FIG. 22 illustrates valve-based recovery of droplets. The droplets can also be barcoded by, for example, using a co-encapsulated bead to facilitate sorting and recovery of the corresponding droplets.

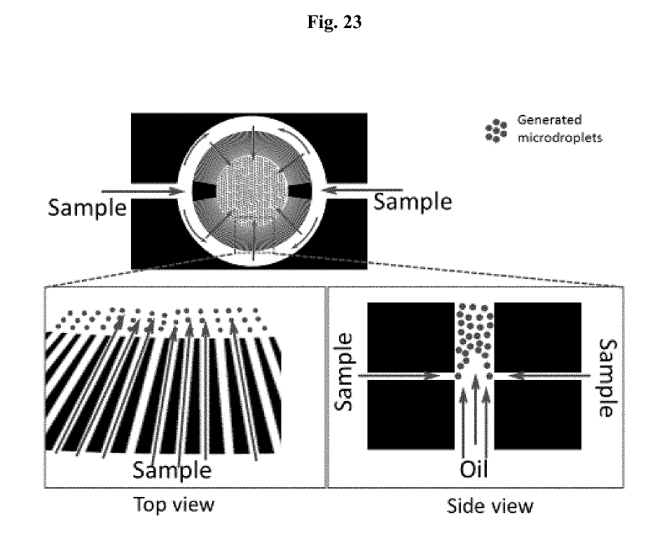

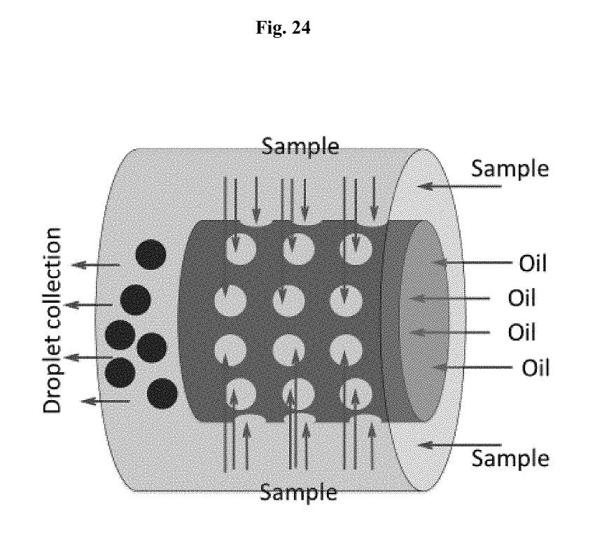

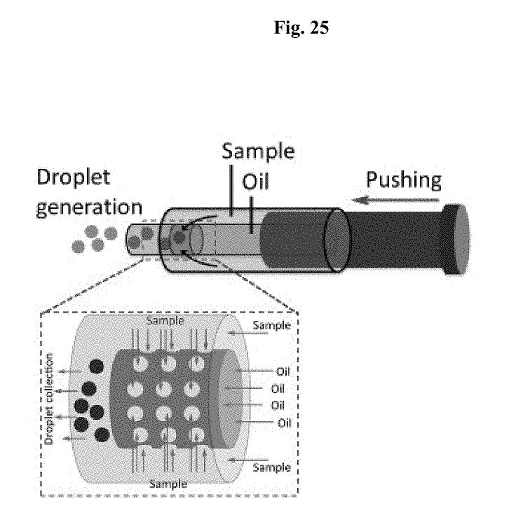

In alternative embodiments, microencapsulated emulsions or droplets can be made using a 2D (FIG. 23) or 3D (FIG. 24)-based high-throughput droplet generation system. For portable systems, microencapsulated emulsions or droplets can be made using a syringe-based high-throughput droplet generator (FIG. 25).

In alternative embodiments, the droplets are formed from a discrete phase with a density greater than the carrier phase and thus droplets are trapped by sinking into the wells.

Alternative exemplary embodiments will be further described with reference to the following examples; however, it is to be understood that these exemplary embodiments are not limited to such examples.

### EXAMPLES

### Example 1: Droplet Microfluidics Fabrication and Setup

### Device Fabrication

The microfluidic device was designed using AutoCAD (Autodesk, San Rafael, Calif., USA) and printed to high-resolution transparency photomasks (CAD/Art Services, Bandon, Oreg., USA). The devices were fabricated from PDMS using standard soft lithography techniques [36]. Four inch silicon wafers were briefly rinsed with 5% hydrofluoric acid (Sigma-Aldrich, St. Louis, Mo., USA) and deionized (DI) water. Prior to spin coating (6NPP-LITE, Laurell Technologies Corporation, USA), wafers were dehydrated in an oven at 95° C. for 10 minutes. Negative photoresist (˜3 g, SU-8 50, MicroChem, Chestech, UK) was then spin-coated (500 rpm for 10 seconds then 3000 rpm for 30 s) onto the wafer. The SU-8 layer was then cured on a hotplate at 65° C. for 5 minutes and at 95° C. for 30 minutes. The cured SU-8 layer was then exposed to UV radiation (14 s, 20 mW/cm2, AB&M INC UV Flood Exposure System) through the photomask and the wafer was subsequently post-baked at 65° C. for 1 minute and 95° C. for 5 minutes. Unexposed SU-8 was removed by soaking in SU-8 developer for 5 minutes. The wafer was then cleaned using isopropyl alcohol, blow dried with filtered nitrogen gas and silanized with perfluorooctyl-trichlorosilane (Sigma-Aldrich, St. Louis, Mo., USA) under vacuum for 3 hours. For fabrication of the devices, PDMS base and curing agent were mixed in a ratio of 10:1 w/w, degassed, poured onto SU8-on-Si wafer masters and fully cured overnight in an oven at 65° C. After thermal curing, the PDMS layer was peeled off the master. Inlet and outlet holes were made with a 1 mm-sized biopsy punch (Kay Industries Co. Tokyo, Japan). PDMS layers were bonded immediately following oxygen plasma treatment and stored overnight before use.

### Example 2: Design of the FDA Device for Ultrahigh-Throughput Droplet Trapping

An example schematic rendering of the FDA device design is shown in FIG. 3. The FDA device consists of two layers of PDMS, one for droplet generation and assembly and the other for droplet trapping. The top layer is designed with a microwell array whose well dimensions can be varied according to the desired droplet size to be trapped. In this work, we used the dimensions (well width×depth) of 30×40, 50×50, 100×50, and 120×50 μm, though other dimensions are also readily used according to the embodiments disclosed herein. Fabricated microwells in the top PDMS layer were characterized by scanning electron microscopy (SEM) as shown in FIG. 3. The diameter of microwells were determined to be 122.5±6.1, 96.7±4.7, 48.6±2.3, and 27.8±1.4 μm, which correspond to a total well number of 9496, 13320, 34560 and 109569, respectively. The bottom PDMS layer was fabricated with a height of 50 μm and contains two aqueous inlets and a single oil inlet whereby the respective fluids are directed to a flow-focusing structure for droplet generation (FIG. 2b, i). The channel width at the flow-focusing structure is 15 μm when the 30 or 50 μm diameter wells were used and 30 μm when the 100 or 120 μm diameter wells were used. After the flow-focusing structure, we included a widened winding channel, which reduces the velocity of the droplets and aides in droplet visualization. The bottom layer also contains a large chamber (18.5 mm wide×37 mm long) which is oriented below the well array. We placed nine large rectangular-shaped resistor structures with long and narrow channels (3 mm long, 200 or 300 μm wide) between them immediately after the entrance of the chamber (FIG. 2a). This provides resistance to flow down the length of the chamber and ensures that droplets spread out across the whole width of the chamber before passing through the narrow channels to the well array (FIG. 2a). We found this helps to ensure compete coverage of the wells. The chamber also contains four pillar structures (1 mm diameter) placed in the central region of the chamber to prevent undesirable bonding of the well array with the bottom of the chamber due to bowing of the PDMS (FIG. 2a). The outlet channels (550 μm wide) are designed at the end of the chamber for collecting excess oil and also to recover the trapped droplets from the FDA device. We also included a waste outlet before the entrance to the chamber to divert undesired droplets such as air, polydisperse, or improperly-sized droplets which often occur at the beginning of device operation from the microwell array. Once generation of the desired droplet size was stable, this waste channel was sealed with a stopper and the droplets were diverted into the chamber for trapping.

### Example 3: Droplet Generation and Manipulation

FIG. 2 shows a step-by-step workflow for the FDA device using dye-containing droplets trapped and released in 120 μm microwells. To operate the device, we initially purged the chamber of air by flowing oil (HFE 7500 without surfactant) through the oil inlet at a flow rate of 10 μl/min for 5 min. Any residual air trapped in the wells was removed by tilting the device at 45° and gently tapping the device with forceps. Aqueous samples were then introduced for droplet generation with the device oriented so that the wells were above the chamber. We generated droplets using HFE 7500+1.8% PFPE-PEG-PFPE surfactant as the oil phase and 10% food coloring dye as the aqueous phase for generating droplet sizes ranging from 20 to 120 μm in diameter by varying the oil and aqueous flow rates (i in FIG. 2b). Initial droplets were diverted into the intermediate waste outlet until the desired droplet size was stably formed. The waste outlet was then sealed with a stopper and the droplets were consequently guided into the chamber, where they spread across the width of the chamber before passing through the narrow channels between the resistor structures (ii in FIG. 2b). The droplets then sequentially filled the wells by floatation due to the density difference between the fluorinated oil and aqueous phase (iii in FIG. 2b). Once the array was completely filled (iv in FIG. 2b), the aqueous inlets were sealed and oil was introduced at a high flow rate (20-30 μl/min) for 10 min to purge the chamber of any extraneous droplets. The trapped droplets were then incubated and analyzed over time (v in FIG. 2b). Subsequently, the droplets were recovered by flipping the device over so that they float out of the wells (vi in FIG. 2b). This simple technique is robust and can be applied to a wide range of droplet sizes. Moreover, it is highly efficient in trapping droplets as can be seen in FIG. 5 with 100% of >14,000 wells analyzed containing a single droplet. We found that with the device dimensions used in this study, we can consistently fill 100% of the microwells with single droplets when they are generated to be 10-20% smaller in diameter compared to the microwells.

### Example 4: Fluorophore Diffusion Between Droplets

For fluorophore diffusion studies, β-gal beads and 500 μM FDG in PBS were introduced into the microfluidic device via respective inlets at a flow rate of 0.5 μL/min, while the oil phase was injected at a flow rate of 15 μL/min. A 2-mm magnetic stir bar was placed inside a 3 mL syringe and was gently mixed by a portable magnetic stirrer (Utah Biodiesel Supply) to prevent settling of the beads. Uniform 55 μm diameter droplets were generated, such that three droplets could fit within 120 μm diameter microwells. Fluorescence intensity of droplets and surrounding oil phase was analyzed under a fluorescence microscope at various time points to monitor the fluorophore-leaking effect between droplets.

### Example 5: Digital Quantification of β-Gal Beads

For the digital quantification of β-gal beads using the FDA device, 25 μm sized-droplets, containing 250 μM FDG with or without a single β-gal bead were trapped within the microfluidic device consisting of 109,569 microwells (30 μm in diameter). After a 10 minute incubation, microscopic images were taken using a 4× objective lens. The experiments were performed in triplicate and the resulting images were analyzed using ImageJ software (ver. 1.48) for quantification of fluorescent droplets.

### Example 6: Real Time Monitoring of Droplet Array

Fluorescent droplets can be monitored in real-time over the cycling using CMOS sensor or full-frame digital camera.

### Example 7: On-Chip Digital PCR and RT-PCR

The FDA device can be used for on-chip and real-time digital PCR (or RT-PCR), that can precisely detect (or quantify) target DNA or RNA sequences, gene mutations and epigenetic modifications, and single-nucleotide polymorphisms (SNP). Droplet-based on-chip and real-time digital PCR can be accomplished using the FDA device by trapping droplets encapsulated with the sample of interest, PCR mixture, and DNA-binding dye or nucleic acid probe (e.g. TaqMan probe). The PCR reaction can be conducted using on-chip thermo cycling.

### Example 8: On-Chip, Digital Isothermal Reaction

The FDA device can also be used with nucleic acid isothermal amplification reactions to detect target DNA or RNA sequences, mutant DNA or RNA, and SNP. This can be used for biological analysis and diagnostics. Some examples of isothermal amplification reactions that may be used include loop-mediated amplification reaction (LAMP), exponential amplification reaction (EXPAR), rolling circle amplification (RCA), strand displacement amplification (SDA), hybridization chain reaction (HCR), nucleic acid sequence based amplification (NASBA), helicase dependent amplification (HDA), nicking enzyme amplification reaction (NEAR), and recombinase polymerase amplification (RPA).

### Example 9: On-Chip, Digital Enzymatic Assay

The FDA device can be used for digital quantification assays in real-time. For this purpose, single enzyme molecule can be encapsulated within droplets with fluorogenic or colorimetric substrates. Fluorescence intensity and number of fluorescent droplets can be monitored in real-time using an on-chip detection system.

### Example 10: HIV Reservoir Detection

The FDA device can be used for quantifying HIV reservoirs in vitro by quantifying a) the total content of cell-associated viral mRNA markers obtained from mononuclear cells, and b) number of cells composing the reservoir at the single-cell level. Cell-associated (CA) HIV-1 mRNA (specifically multiply spliced (ms) tat/rev) can be used here as an indicator of residual viral replication and the size of HIV reservoir because they directly correlate with the reactivation of latent reservoir in vivo. Isolated peripheral blood mononuclear cells (PBMCs) can be encapsulated in droplets at the single-cell level after stimulation with an agent such as phorbol 12-myristate 13-acetate plus ionomycin (PMA/I) to induce viral mRNA expression. Levels of HIV rev/tat expression per cell and absolute number of HIV reservoir cells can be determined using the FDA-based digital RT-PCR.

### Example 11: Circulating Tumor Cells and Tumor Free DNA/RNA Detection

The FDA device can be used to detect circulating tumor cells (CTCs) and tumor cell-free DNA (or RNA) in the blood. For monitoring CTCs in the blood, red blood cells will be lysed and PBMCs will be encapsulated at the single-cell level per droplet. Single-cell PCR, single-cell RT-PCR, single-cell isothermal DNA (or RNA) amplification (mentioned in example 8), and proximity ligation for isothermal amplification (or DNA strand displacement) can be used to generate a fluorescent signal in droplets that contain single-CTC. For the circulating tumor cell-free DNA (or RNA), plasma sample or isolated DNA (or RNA) can also be analyzed in the FDA device in a similar manner.

### Example 12: On-Chip, Cell Culture and Detection

Cells can be encapsulated at the single-cell level per droplets and can be grown within droplets to increase the population for:

a) On-chip colony forming unit (CFU) assay to quantity the number of cells. The number of droplets that contain a bacteria colony, can be visualized by staining cells via colorimetric or fluorescence dye; or

b) Identification (or profiling) of the cells by monitoring protein secretion. Secreted proteins can be monitored using enzymatic activity assays with fluorogenic/colorimetric substrates or antibody-based proximity ligation to induce isothermal amplification within droplets. The population of cells, secreting a protein of interest, can be quantified in real-time using the FDA device.

### Example 13: On-Chip, Cell-Cell Interaction and Cell-Fusion

Using the FDA device, single cells can be encapsulated into droplets and trapped into trapping structures such as microwells. Other types of single cell can be encapsulated into droplets and arranged neighboring previously trapped droplets. By using chemical reagents (such as alcohol solution) or electric fields, droplet fusion or diffusion of molecules between droplets can be controlled for various purposes as described below:

a) To monitor cell-cell communication at the single-cell level. Two different types of cells (e.g. a colon cancer cell and mesenchyme stem cell) can be encapsulated in separate droplets and then droplets will be trapped in neighboring trapping structures. Chemical reagents or an electric field can be used to induce permeabilization of molecules between droplets.

b) Cell-cell fusion for hybridoma screening. Two different droplets can be generated, one containing a myeloma (B cell cancer) and the other droplet containing an antibody-producing B cell. Then, the two different droplets can be trapped in neighboring trapping structures such that the droplets are in contact. The two droplets can be merged (fused) and cell-fusion can be controlled by osmotic pressure or electric field.

### Example 14: On-Chip Screening for Receptor-Ligand Interaction and Therapeutic Screening

To monitor receptor-ligand interactions e.g., protein-protein interaction using the FDA device, two different proteins can be encapsulated in separate droplets and then the two droplets can be trapped in neighboring trapping structures. Trapped droplets can be merged by the methods as described in Example 13. For monitoring protein-protein interaction, FRET, life-time imaging, or fluorescence polarization can be integrated in the FDA device.

For the inhibitor screening, protein A, protein B, and a library (small molecule, DNA, peptide, antibody or protein) can be encapsulated within separated droplets. Protein A-containing droplets and library-containing droplets can be merged first by activation of an electrode that is located between droplets (see FIG. 20) and then the other electrode can control merging of the other droplet, containing protein B, with previously merged droplet. Inhibitory effect can be monitored using FRET, life-time imaging, or fluorescence polarization.

### Example 15: On-Chip In Vitro Evolution, Selection and Screening

The FDA device can be used for in vitro evolution, selection and screening. An aptamer library can be compartmentalized within picoliter droplets and trapped within microwell structures. Then the target molecules can also be encapsulated and droplets can be trapped next to the library containing droplets. Two droplets can be merged by the method described above (example 13) and target-aptamer interactions can be used to trigger a fluorescence signal for example by triggering isothermal amplification reaction as describe in example 8.

### REFERENCES

Hatch, A. C.; Fisher, J. S.; Tovar, A. R.; Hsieh, A. T.; Lin, R.; Pentoney, S. L.; Yang, D. L.; Lee, A. P. 1-million droplet array with wide-field fluorescence imaging for digital per. Lab on a chip 2011, 11, 3838-3845.
Huebner, A.; Bratton, D.; Whyte, G.; Yang, M.; deMello, A. J.; Abell, C.; Hollfelder, F. Static microdroplet arrays: A microfluidic device for droplet trapping, incubation and release for enzymatic and cell-based assays. Lab on a chip 2009, 9, 692-698.
Schmitz, C. H. J.; Rowat, A. C.; Koster, S.; Weitz, D. A. Dropspots: A picoliter array in a microfluidic device. Lab on a chip 2009, 9, 44-49
U.S. Pat. No. 8,597,486 B2; U.S. Pat. No. 8,034,628 B2; US 2011/0190146 A1
U.S. Pat. No. 8,691,147 B2; U.S. Pat. No. 8,883,513 B2; US 2011/0092376 A1
EP 2 703 497 A1; U.S. Pat. No. 8,730,479 B2

A number of embodiments have been described. Nevertheless, it will be understood that various modifications may be made without departing from the spirit and scope of the exemplary embodiments provided herein. Accordingly, other embodiments are within the scope of the following claims.

1: A high throughput, multiplexed system or device, or method, for detecting and/or quantifying a chemical, biological, physiological or pathological analyte, or a single molecule or a single cell, or a chemical or a biochemical reaction; or recognition of a cell or molecule, using a floating droplet array (FDA) system integrated with use of a sensing element or reporter or a fluorogenic reaction, comprising:
(a) providing a sensor or sensing reaction that involves a small molecule, peptide, protein, nucleic acid, enzyme, antibody, cell, or chemical agent capable of detecting a target of interest and producing a signal readout;
(b) providing a floating droplet array system, droplet microfluidics system or microdroplet-manipulating device or system;
(c) providing a chemical, biological or an environmental sample containing a target of interest such as a small molecule, an aptamer, a metabolite, peptide, protein, nucleic acid, or cell; and
(d) encapsulating the chemical, biological or environmental sample into a plurality of positive microdroplets, trapping the positive microdroplets into trapping structures, and processing the positive microdroplets comprising the encapsulated chemical, biological or environmental sample in the positive droplet microfluidics system or microdroplet-manipulating device, and detecting the presence of a fluorophore signal, or fluorescence, in each positive microdroplet in the device,
wherein detection of a fluorophore signal or fluorescence in a microdroplet indicates the presence of the target molecule in the positive microdroplet, and the sample,
and optionally further comprising:
(d) sorting individually or collectively the positive microdroplets for downstream analysis including sequencing technologies.

2: A high throughput, multiplexed system or device, or method, for detecting and/or quantifying one or multiple different types of a chemical, a biological, a physiological or a pathological analyte, or one or multiple different types of a single molecule or a single cell, or a chemical or a biochemical reaction, or recognition of a cell or a molecule, comprising use of a floating droplet array system,
wherein optionally the floating droplet array system is in real-time,
and optionally one of the signals in the said multiplex assay serves for reference or normalization purposes.

3. (canceled)

4: The high throughput, multiplexed system or device, or method, of claim 1, wherein the cell is a mammalian cell, a circulating tumor cell, a circulating melanoma cell, a B cell, a hybridoma cell, a T cell, a Chimeric Antigen Receptor (CAR) T cell (CAR-T cell), a fungal cell, virus or a bacterial cell, a stem cell, a differentiated cell, an engineered cell;
and optionally a heterogeneous cell population can be partitioned and encapsulated into droplets and characterized, manipulated and sorted at a single-cell level; and optionally a droplet can contain one, two, three, four or more of the same type of cell or different types of cells.

5: The high throughput, multiplexed system or method of claim 1, wherein:
(a) the droplet microfluidics system can generate: picoliter droplets or droplets of between about 2 μm to 999 μm in diameter;
(b) 100 to 100 billion droplets can be immobilized and analyzed in the droplet array;
(c) droplets are composed of one or many sub-phases as single or multiple emulsions.

6-7. (canceled)

8: The high throughput, multiplexed system or device, or method, of claim 1, wherein:
the biological sample comprises a blood, serum, saliva, tear, urine, tissue, or CSF sample from an individual, a patient or an animal, as well as non-biological samples including food, water and environmental samples;
(b) the single molecule is a nucleic acid, a nucleic acid point mutation, or a single-nucleotide polymorphism (SNP), ribonucleic acid or a nucleic acid biomarker, and optionally the nucleic acid biomarker is for a cancer, optionally a breast cancer;
(c) the single molecule is a protein, a lipid, a carbohydrate, a polysaccharide, a small molecule or a metal;
(d) the single cell is a bacteria, a fungi, a virus, a mammalian cell, or a fused cell; and optionally in alternative embodiments, the encapsulated cells can be cultured in droplets without significantly losing their viability from hours to 7 days;
(e) the encapsulated cell(s) or molecule(s) produce a fluorescent signal upon a chemical or biological reaction, and optionally a B cell produces target antibodies or a Chimeric Antigen Receptor (CAR) T cell (CAR-T) kills a cancer cell; or
(f) the sensor or reaction comprises a DNA strand displacement strategy, a proximity ligation assay, a binding induced DNA assembly assay, a PCR or RT-PCR reaction, an enzyme reaction, a fluorescent dye or protein, or a fluorogenic reaction; and optionally a reagent or reagents are co-encapsulated with analytes or samples at the beginning or optionally introduced sequentially, optionally co-encapsulated by droplet-droplet fusion.

9-13. (canceled)

14: The high throughput, multiplexed system or device, or method, of claim 1, further comprising detecting and/or quantifying the chemical, biological, physiological or pathological analyte, or single molecule or single cell integrated with a detection system comprising the use of an embedded APD (avalanche photodiode), photomultiplier tube (PMT), digital camera, charge-coupled device (CCD) or complementary metal-oxide semiconductor (CMOS) sensor in a high throughput manner.

15: The high throughput, multiplexed system or device, or method, of claim 1, wherein:
(a) the throughput, multiplexed system is engineered to comprise one or any of: desirable portability, automating fluid handing, and integrating electronics including a diode laser, LED panel, light source, operating, and/or data analyzing software, display with fluorescence microscopy, embedded APD (avalanche photodiode), photomultiplier tube (PMT), digital camera, charge-coupled device (CCD), complementary metal-oxide semiconductor (CMOS) sensor; or
(b) further comprising disposable microfluidic “cartridges,” permitting multiplex and rapid detection of multiple types of targets simultaneously, and optionally the high throughput, multiplexed system or device is fully automated, or is fabricated as an all-in-one system or with modular components, or is linked to an electronic device, e.g., a portable device, e.g., a smart phone and/or a Bluetooth, or is integrated with a portable temperature controller for point-of-care applications, wherein optionally the portable temperature controller is a Peltier-based thermocycler.

16. (canceled)

17: A high-throughput system comprising trapping structures of various sizes or shapes for immobilizing droplets of various compositions in a spatially controlled, defined, and parallel format.

18: The high-throughput system of claim 17, comprising:
(a) trapping droplets into trapping structures, whereby droplets float or sink into trapping structures due to density differences between the dispersed and continuous phases;
and optionally the droplets may be recovered by reorienting the device;
and optionally the droplets may be fused or merged or split in step-wise processes to accommodate chemistries or stimulations or media change in sequential steps;
(b) a multilayer microfluidic device whereby droplets are trapped in a region above or below the main flow stream;
(c) guiding structures such as tracks, pillars, or narrow channels which guide droplets to the trapping structures and ensure complete and efficient coverage of the trapping structures; or
(d) inlets or outlets to divert droplets away or to the droplet trapping structures and/or other channels or chambers.

19-21. (canceled)

22: The high-throughput system of claim 17:
(a) further comprising indexing droplets based on one or many spatial or temporal variables;
(b) wherein the droplets are indexed based on uniquely barcoded beads, a nucleic acid barcode, a fluorophore, an organic or an inorganic dye barcode, or a colorimetric barcode;
(c) wherein the high-throughput system is integrated with:
(i) a data acquisition hardware or a software, a data analysis software, a user interface, or a computer or a mobile device application, or
(ii) integrated with a sorting element or elements for retrieving a plurality of or individual droplets, whereby optionally the sorting element comprises an electrode, a pneumatic valve, a microfluidic controlled valve, a laser, a microneedle, a magnetic field or an acoustic-based droplet retrieval system;
(d) wherein sorted or retrieved droplets are analyzed using downstream methods for their contents,
whereby optionally the downstream methods comprise sequencing, next-generation sequencing (NGS), or
the sorted or retrieved droplets are analyzed using pyrosequencing or massively parallel signature sequencing, or analyzed using single-cell sequencing techniques; or
(e) wherein the high-throughput system serves as a research or discovery tool to characterize, manipulate, screen, and/or sort immunological agents including B cells, plasma cells, hybridomas, antibodies, monoclonal antibodies, nanobodies, antibody-drug conjugates, T cells, a Chimeric Antigen Receptor (CAR) T cell (CAR-T), native or engineered cells; optionally the presence or production of the said immunological agent(s) or when the said immunological agent(s) activate, inhibit or modulate a biological molecule, signal or event in the droplets produce a detectable signal readout; optionally the said signal readout can indicate or quantify the presence, or binding or biological functions of the said immunological agent(s).

23-27. (canceled)

28: A high-throughput droplet generation module, whereby droplets are formed at high throughput using droplet-generating junctions comprising:
(a) a dispersed phase which flows through a plurality of channels in a radial direction prior to dispersion;
(b) a carrier phase that flows through one or many channels in a direction perpendicular to the radially flowing phase that is to be dispersed; and
(c) droplets generated as the carrier phase comes into contact with an immiscible dispersed phase in the arrangement described in (a) and (b).

29: The high-throughput system of claim 28, comprising a stacked, 3D arrangement of droplet-generating junctions such that droplet generation can occur at many junctions simultaneously.

30: The high-throughput system of claim 28 in which the droplet-generating module is integrated within a portable handheld fluidic device, such as a syringe.

31: The high-throughput system of claim 28, whereby the driving pressure for fluid flow can be generated by hand using a force-transferring device such as a plunger.
